(19) United States
(12) Patent Application Publication
(10) Pub. No.: US 2023/0377755 A1
(43) Pub. Date: Nov. 23, 2023

(54) Construction Method For Treatment Reactivity Predicating Model Of Hepatocellular Carcinoma Based on Gene Expression Quantity

(71) Applicant: The First Affiliated Hospital of Sun Yat-Sen University, Guangzhou (CN)

(72) Inventors: Jiaping Li, Guangzhou (CN); Yiyang Tang, Guangzhou (CN)

(21) Appl. No.: 18/313,332

(22) Filed: May 6, 2023

(30) Foreign Application Priority Data
May 20, 2022 (CN) 202210559298.4

(51) Int. Cl. G16H 50/30

(52) U.S. Cl. CPC G16H 50/30

(57) ABSTRACT

The present disclosure provides a construction method for a treatment reactivity predicating model of hepatocellular carcinoma based on a gene expression quantity. According to the method of the present disclosure, samples are selectively distinguished through the expression quantity of the genes, and the reaction of patients to TACE treatment can be more accurately predicted.

### TECHNICAL FIELD

The present disclosure relates to the field of medical technologies and in particular to a construction method for a treatment reactivity predicating model of hepatocellular carcinoma based on a gene expression quantity.

### BACKGROUND

Transarterial chemoembolization (TACE) is currently recognized as the most common non-surgical treatment for liver cancer, and it is widely used in patients with stage IIb-IIIa liver cancer in China. Chemoembolization can be achieved by injecting chemotherapy drugs and embolic agents or drug-loaded microspheres after inserting a catheter selectively into a tumor-feeding artery. Because of the heterogeneity of intermediate-stage hepatoma and the widespread use of TACE, patients' responses and effectiveness vary greatly. As a result, it is critical to screen patients who respond well to TACE for appropriate treatment.

The current scoring systems for predicting the postoperative effects of TACE mainly rely on indicators of routine clinical measurement for evaluation. Since the existing prediction of TACE reactivity is more dependent on indicators that are readily available in clinical practice, the accuracy of the prediction is greatly reduced, although it is simple and convenient, but also impairs the reliability and validity. Furthermore, the majority of current indicators are hepatoma-specific, rather than TACE-specific.

To address the aforementioned issues, we propose a method for predicting the treatment reactivity model of hepatocellular carcinoma based on gene expression quantity.

### SUMMARY

The present disclosure provides a construction method for a treatment reactivity predicating model of hepatocellular carcinoma based on a gene expression quantity, in order to solve at least one of the problems in the background art.

A construction method for a treatment reactivity predicating model of hepatocellular carcinoma based on a gene expression quantity, including the following steps of:
step 1: obtaining tumor tissue samples of hepatocellular carcinoma patients in the GSE104580 database via percutaneous biopsy prior to TACE, and determining the transcriptome expression quantity by gene chip sequencing of samples;
step 2: randomly dividing tumor tissue samples from hepatocellular carcinoma patients into a training set and a verification set, dividing the training set into a reaction group and a non-reaction group based on patients' reactivity to TACE treatment, and identifying response-related genes by using a differential expression gene method, then building a model using five modeling methods: LASSO (Least Absolute Shrinkage and Selection Operator)-logistic regression, random forest, xgboost-random forest, multi-layer perceptual network, and support vector machine, comparing each model by a Receiver Operating Characteristic (ROC) curve and finally determining a support vector machine model based on gene expressions and corresponding weight coefficients; and
step 3: dividing patients into a TACE reaction group and a TACE non-reaction group based on a median risk score as a threshold, performing statistical analysis and comparison on TACE efficacy difference of patients between the two groups and evaluating and predicting the best predictive model of treatment reactivity of hepatocellular carcinoma.

As a preferred embodiment, the 10 related genes are AQP1, FABP4, HERC6, LOX, PEG10, S100A8, SPARCL1, TIAM1, TSPAN8, and TYRO3, respectively.

As a preferred embodiment, in step 3, the risk score is calculated by the following formula: X=A1*B1+A2*B2+ . . . +A10*B10, where B1, B2, . . . and B10 are expression levels of 10 genes included in the model, and A1, A2, . . . and A10 are weight coefficients of 10 genes calculated by LASSO regression.

As a preferred embodiment, the construction method for the treatment reactivity predicating model of the hepatocellular carcinoma based on the gene expression quantity further includes step 4: testing the predictive ability of the TACE treatment reactivity model of hepatocellular carcinoma in the external validation set.

As a preferred embodiment, in step 4, the risk score of each sample in the validation set is calculated by the same formula.

As a preferred embodiment, after the risk score of each sample in the validation set is calculated, patients are divided into a TACE reaction group and a TACE non-reaction group based on a median risk score in the training set as the boundary value, and analysis is carried out on the overall survival time between the two groups for statistical differences.

As a preferred embodiment, in step 4, the number of tumor tissue samples of the patients with hepatocellular carcinoma is not less than 100.

As a preferred embodiment, in steps 3 and 4, the step of evaluating the performance of the predictive TACE treatment reactivity model of hepatocellular carcinoma is as follows:
evaluating the performance of the predictive model for TACE treatment reactivity of hepatocellular carcinoma by multivariate COX proportional hazard regression analysis and ROC curve.

Beneficial effects: from a medical standpoint, the method of the present disclosure selectively distinguishes samples based on the expression quantity of 10 genes, which can more accurately predict patient response to TACE treatment and guide the next treatment for patients. TACE specificity solves the problem of TACE prediction accuracy being greatly reduced because the existing prediction of TACE reactivity is more dependent on clinically available indicators, thereby increasing the reliability and validity.

### BRIEF DESCRIPTION OF THE DRAWINGS

FIG. 1 shows a step diagram of a method of the present disclosure.

### DETAILED DESCRIPTION OF THE EMBODIMENTS

The present disclosure is further described in detail in combination with the accompanying drawings. These drawings are simplified schematics that only composition related to the present disclosure and only illustrate the basic structure of the present disclosure in a schematic manner. For those skilled in the art, the specific meanings of the above terms in the present disclosure can be understood in accordance with specific cases.

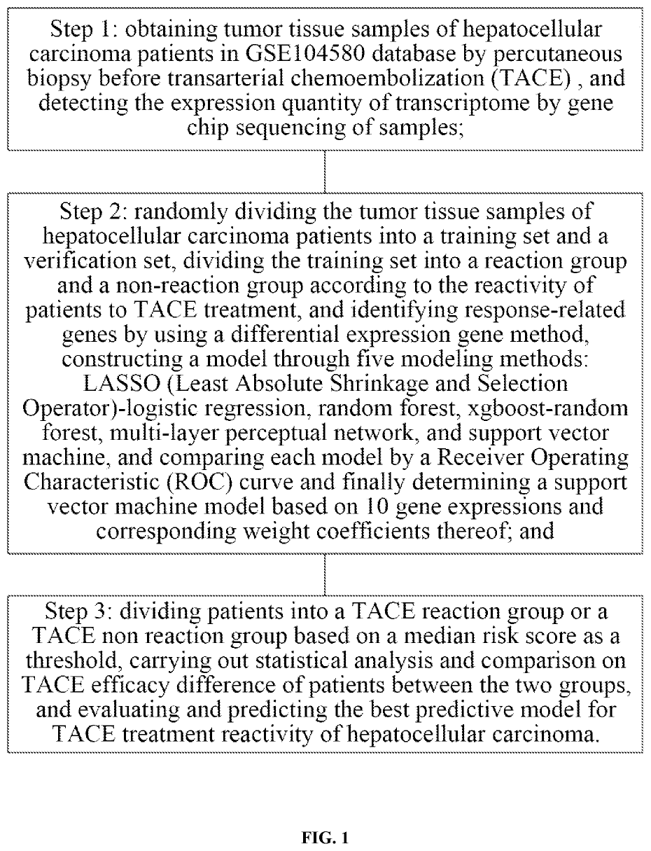

As shown in FIG. 1, the present disclosure provides a construction method for a treatment reactivity predicating model of hepatocellular carcinoma based on a gene expression quantity, including the following steps:
step 1: obtaining tumor tissue samples of hepatocellular carcinoma patients in the GSE104580 database via percutaneous biopsy prior TACE, and determining the transcriptome expression quantity by gene chip sequencing of samples;
step 2: randomly dividing tumor tissue samples from hepatocellular carcinoma patients into a training set and a verification set, dividing the training set into a reaction group and a non-reaction group based on patients' reactivity to TACE treatment, and identifying response-related genes by using a differential expression gene method, then building a model using five modeling methods: LASSO-logistic regression, random forest, xgboost-random forest, multi-layer perceptual network, and support vector machine, comparing each model by a ROC curve and finally determining a support vector machine model based on 10 gene expressions and corresponding weight coefficients thereof; and the 10 related genes being, respectively, AQP1, FABP4, HERC6, LOX, PEG10, S100A8, SPARCL1, TIAM1, TSPAN8 and TYRO3;
step 3: dividing patients into a TACE reaction group and a TACE non-reaction group based on the median risk score as the threshold, performing statistical analysis and comparison on TACE efficacy difference of patients between the two groups, and evaluating and predict the best predictive model of treatment reactivity of hepatocellular carcinoma. In step 3, the risk score is calculated by the following formula: X=A1*B1+A2*B2+ . . . +A10*B10, where B1, B2, . . . and B10 are expression levels of 10 genes included in the model, and A1, A2, . . . and A10 are weight coefficients of 10 genes calculated by LASSO regression; and
step 4: testing the predictive ability of the TACE treatment reactivity model of hepatocellular carcinoma in the external validation set, where a risk score of each sample in the validation set is calculated using the same formula; and after calculating the risk score of each sample in the validation set, patients are divided into a TACE reaction group and a TACE non-reaction group with the median risk score in the training set as the boundary value, and the overall survival time between the two groups is analyzed for statistical differences, with a minimum of 100 hepatocellular carcinoma tumor tissue samples.

In steps 3 and 4, the step of evaluating the performance of the predictive model for TACE treatment reactivity of hepatocellular carcinoma is; evaluating the performance of the predictive model for TACE treatment reactivity of hepatocellular carcinoma by multivariate COX proportional hazard regression analysis and ROC curve.

From the medical standpoint, the present disclosure's method selectively distinguishes samples based on the expression quantity of 10 genes, which can more accurately predict patient response to TACE treatment and guide patients' next treatment. TACE specificity solves the problem of TACE prediction accuracy being greatly reduced because the existing prediction of TACE reactivity is more dependent on clinically available indicators, thereby increasing the reliability and validity.
10 genes of the present disclosure and corresponding coefficients thereof

| Genes | Occurrence | Ranking sum | Average rank | Coefficient |
|---|---|---|---|---|
| Genes | Occurrence | Ranking sum | Average rank | Coefficient |
| TSPAN8 | 10/10 | 31 | 3.1 | 0.053 |
| SPARCL1 | 10/10 | 70 | 7 | −0.098 |
| S100A8 | 10/10 | 128 | 12.8 | 0.064 |
| AQP1 | 10/10 | 212 | 21.2 | −0.069 |
| TYRO3 | 9/10 | 135 | 15 | 0.084 |
| TIAM1 | 9/10 | 171 | 19 | −0.054 |
| HERC6 | 9/10 | 224 | 24.9 | −0.054 |
| LOX | 9/10 | 306 | 34 | 0.065 |
| FABP4 | 8/10 | 178 | 22.2 | −0.033 |
| PEG10 | 8/10 | 194 | 24.2 | 0.027 |

TSPAN8: Tetraspanin 8
SPARCL1: SPARC like 1
S100A8: S100 calcium binding protein A8;
AQP1: Aquaporin 1;
TYRO3: TYRO3 protein tyrosine kinase;
TIAM1: TIAM Rac1 associated GEF 1;
HERC6: HECT and RLD domain containing E3 ubiquitin protein ligase family member
6;
LOX: Lysyl oxidase;
FABP4: Fatty acid binding protein 4;
PEG10: Paternally expressed 10.

In the description of the present disclosure, the description of reference terms “one embodiment”, “certain embodiments”, “schematic embodiments”, “examples”, “specific examples”, or “some examples” means that specific features, structures, materials, or characteristics described in conjunction with the embodiments or examples are included in at least one embodiment or example of the present disclosure. In the description, the schematic expressions of the above terms do not always refer to the same embodiments or examples. Furthermore, the specific features, structures, materials or characteristics described may be combined in any or more embodiments or examples in an appropriate number.

The preceding illustrates and describes the fundamental principles, key features and advantages of the present disclosure. For those skilled in the art, it is obvious that the present disclosure is not limited to the details of the exemplary embodiments discussed above, and that it can be realized in other specific forms without departing from the spirit or basic features of the present disclosure. As a result, the embodiments should be regarded as exemplary and non-restrictive from any perspective. The appended claims, rather than the above description, define the scope of the present disclosure. Therefore, all changes within the meaning and scope of the equivalent elements of the claims in the present disclosure are intended to be included. Any reference sign in the claims is not intended to limit the scope of the claims.

In addition, while the Description is organized according to the embodiments, not every embodiment contains only an independent technical solution. This representation of the Description is only for the sake of clarity. Those skilled in the art should consider the Description as a whole, and the technical solutions in each embodiment can be properly combined to form other embodiments that those skilled in the art can understand.

What is claimed is:

1. A construction method for a treatment reactivity predicating model of hepatocellular carcinoma based on a gene expression quantity, comprising the following steps:
step 1: obtaining tumor tissue samples of hepatocellular carcinoma patients in GSE104580 database by percutaneous biopsy prior to transarterial chemoembolization (TACE), and detecting the expression quantity of transcriptome by gene chip sequencing of samples;
step 2: randomly dividing the tumor tissue samples of hepatocellular carcinoma patients into a training set and a verification set, dividing the training set into a reaction group and a non-reaction group based on patients' reactivity to TACE treatment, and identifying response-related genes by using a differential expression gene method, then building a model using five modeling methods: LASSO (Least Absolute Shrinkage and Selection Operator)-logistic regression, random forest, xgboost-random forest, multi-layer perceptual network, and support vector machine, comparing each model by a Receiver Operating Characteristic (ROC) curve and finally determining a support vector machine model based on 10 gene expressions and corresponding weight coefficients thereof; and
step 3: dividing patients into a TACE reaction group and a TACE non-reaction group based on a median risk score as a threshold, performing statistical analysis and comparison on TACE efficacy difference of patients between the two groups and evaluating and confirming the best predictive model for hepatocellular carcinoma TACE treatment reactivity.

2. The construction method for the treatment reactivity predicating model of the hepatocellular carcinoma based on the gene expression quantity according to claim 1, wherein the 10 related genes are AQP1, FABP4, HERC6, LOX, PEG10, S100A8, SPARCL1, TIAM1, TSPAN8, and TYRO3.

3. The construction method for the treatment reactivity predicating model of the hepatocellular carcinoma based on the gene expression quantity according to claim 2, wherein in step 3, the risk score is calculated using the following formula: X=A1*B1+A2*B2+ . . . +A10*B10, where B1, B2, . . . and B10 are expression levels of 10 genes included in the model, and A1, A2, . . . and A10 are weight coefficients of 10 genes calculated using LASSO regression.

4. The construction method for the treatment reactivity predicating model of the hepatocellular carcinoma based on the gene expression quantity according to claim 3, further comprising:
step 4: testing a predictive ability of hepatocellular carcinoma TACE treatment reactivity predicting model in the external validation set.

5. The construction method for the treatment reactivity predicating model of the hepatocellular carcinoma based on the gene expression quantity according to claim 4, wherein in step 4, a risk score of each sample in the validation set is calculated by the same formula.

6. The construction method for the treatment reactivity predicating model of the hepatocellular carcinoma based on the gene expression quantity according to claim 5, wherein after calculating the risk score of each sample in the validation set, patients are divided into a TACE reaction group and a TACE non-reaction group based on the median risk score in the training set as the boundary value, and statistical analysis is performed on the overall survival time between the two groups.

7. The construction method for the treatment reactivity predicating model of the hepatocellular carcinoma based on the gene expression quantity according to claim 6, wherein in step 4, the number of tumor tissue samples from hepatocellular carcinoma patients is greater than 100.

8. The construction method for the treatment reactivity predicating model of the hepatocellular carcinoma based on the gene expression quantity according to claim 7, wherein in steps 3 and 4, the step of evaluating the performance of the predictive model for TACE treatment reactivity of hepatocellular carcinoma is; evaluating the performance of the predictive model for TACE treatment reactivity of hepatocellular carcinoma by multivariate COX proportional hazard regression analysis and ROC curve.
